(19) United States
(12) Patent Application Publication
(10) Pub. No.: US 2026/0263793 A1
(43) Pub. Date: Sep. 10, 2026

(54) METHODS OF IMPROVING PLATELET FUNCTION OF BLOOD DONORS FOR HEMOPHILIAC SUBJECTS

(71) Applicant: The Feinstein Institutes for Medical Research, Manhasset, NY (US)

(72) Inventors: Jared M. HUSTON, Old Field, NY (US); Kevin J. TRACEY, Old Greenwich, CT (US)

(21) Appl. No.: 19/663,245

(22) Filed: Apr. 29, 2026

Related U.S. Application Data
(62) Division of application No. 18/355,401, filed on Jul. 19, 2023, now Pat. No. 12,649,059.
(60) Provisional application No. 63/395,775, filed on Aug. 5, 2022.

(51) Int. Cl. A61N 1/36

(52) U.S. Cl. CPC A61N 1/36014

(57) ABSTRACT

Methods of improving platelet function of blood donors for hemophiliac subjects. Methods may include stimulating the vagus nerve of the blood donor to increase calcium uptake of the donor's platelets. The blood donor may not have been administered a clotting factor within a certain period.

### CLAIM OF PRIORITY

This patent application is a divisional of U.S. patent application Ser. No. 18/355,401, titled “METHODS FOR REDUCING BLEEDING IN HEMOPHILIA BY VAGUS NERVE STIMULATION TO PRIME PLATELETS,” filed on Jul. 19, 2023, now U.S. Patent Application Publication No. 2024/0042201, which claims priority to U.S. Provisional Patent Application No. 63/395,775, titled “METHODS FOR REDUCING BLEEDING IN HEMOPHILIA BY VAGUS NERVE STIMULATION TO PRIME PLATELETS,” filed on Aug. 5, 2022, each of which are herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Deficiency of coagulation factor VIII in hemophilia A disrupts clot formation and prolongs bleeding after injury. While the current mainstay of therapy is recurrent infusion of factor VIII concentrates, inhibitor antibodies often render these ineffective, mandating costly thrombotic therapies with higher morbidity and mortality. Preclinical evidence shows electrical vagus nerve stimulation accelerates clot formation and increases clot deposition to reduce traumatic bleeding.

### SUMMARY OF THE DISCLOSURE

As described herein, vagus nerve stimulation augments clotting at the site of injury without precipitating systemic thrombosis, and may reduce traumatic bleeding in hemophilia A subjects. In both arterial hemorrhage and thrombosis models, we observed that vagus nerve stimulation bypasses the factor VIII deficiency of hemophilia A to decrease blood loss and accelerate clot formation. Vagus nerve stimulation targets acetylcholine-producing T lymphocytes in spleen and platelet alpha 7 nicotinic acetylcholine receptors (a7nAChR) to increase calcium uptake and enhance alpha granule release and surface expression of P-selectin. Splenectomy or genetic deletion of T cells or a7nAChR abolishes vagus nerve-mediated control of platelet calcium uptake and activation, thrombus formation, and bleeding time. These results reveal an endogenous vagus nerve pathway harnessing platelets to augment clot formation, which restores hemostasis in hemophilia A in lieu of clotting factor administration. Vagus nerve stimulation warrants clinical study as a potential therapy for coagulation disorders and surgical or traumatic bleeding.

In particular, vagus nerve stimulation may be applied as described herein to reduce traumatic bleeding in hemophilia A subjects that have not received a clotting factor. For example, subject's may have not received a clotting factor within at least the last 48 hours, the last 36 hours, the last 24 hours, the last 18 hours, the last 12 hours, the last 10 hours, the last 8 hours, the last 6 hours, the last 4 hours, or the last 2 hours. In the absence of an administered clotting factor, vagus nerve stimulation may work surprisingly well, even at relatively low stimulation “doses”.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A shows vagus nerve stimulation reduces traumatic blood loss in hemophilia A mice. a, Circulating factor VIII activity in wild-type and factor VIII deficient (hemophilia A) mice. Data are presented as mean±s.e.m. (n=7 mice). ****, p<0.0001 vs. control.

FIG. 1B shows Hemophilia A mice received rFVIII (Advate®, 200 U/kg, r.o.) or vehicle before tail transection. Data are presented as mean±s.e.m. (n=7-8 mice per group). *, p<0.05 vs. sham.

FIG. 1C, Hemophilia A mice received vagus nerve stimulation or sham stimulation before tail transection. Data are presented as mean±s.e.m. (n=6-9 mice per group). *, p<0.05 vs. wild type. **, p<0.01 vs. sham.

FIG. 1D, Hemophilia A mice received vagus nerve stimulation or sham stimulation before blood collection to determine factor VIII activity. Data are presented as mean±s.e.m. (n=8-11 mice per group). p=NS vs. sham.

FIG. 1E, Hemophilia A mice received vagus nerve stimulation or sham stimulation before carotid artery injury. Data are presented as mean #s.e.m. (n=6 mice per group). ****, p<0.0001 vs. wt. ****, p<0.0001 vs. sham.

FIG. 2A shows vagus nerve stimulation harnesses choline acetyltransferase-expressing T cells in spleen to stimulate platelets via a7nAChR. a, C57BL6/J mice underwent splenectomy or sham splenectomy followed by vagus nerve stimulation or sham stimulation before tail transection. Data are presented as mean±s.e.m. (n=6-9 mice per group). **, p<0.01 versus sham.

FIG. 2B, Wild-type BALB/c or T lymphocyte deficient (Foxn1nu) mice received vagus nerve stimulation or sham stimulation before tail transection. Data are presented as mean±s.e.m. (n=4-6 mice per group). *, p=0.05 vs. sham.

FIG. 2C, T lymphocyte deficient (Foxn1nu) mice were reconstituted with ChAT-eGFP+ or ChAT-eGFP-cells followed by vagus nerve stimulation or sham stimulation before tail transection. Data are presented as mean±s.e.m. (n=5-8 mice per group). **, p<0.01 vs. sham.

FIG. 2D, Platelets from uninjured C57BL6/J mice receiving vagus nerve stimulation or sham stimulation were transferred into naïve animals before tail transection. Data are presented as mean±s.e.m. (n=8-9 mice per group). *, p<0.05 vs. sham.

FIG. 2E, Wild-type or a7nAChR-deficient mice received vagus nerve stimulation or sham stimulation before tail transection. Data are presented as mean±s.e.m. (n=4-5 mice per group). *, p<0.05 vs. sham.

FIG. 2F, Wild-type or a7nAChR-deficient mice received vagus nerve stimulation or sham stimulation before carotid artery injury. Data are presented as mean±s.e.m. (n=5 mice per group). *, p<0.05 vs. sham.

FIG. 2G, Wild-type or a7nAChR-deficient mice received nicotine or vehicle before tail transection. Data are presented as mean±s.e.m. (n=4-8 mice per group). **, p<0.01 vs. vehicle control.

FIG. 2H, a7nAChR deficient mice were reconstituted with platelets from wild-type or a7nAChR-deficient mice followed by treatment with nicotine or vehicle before tail transection. Data are presented as mean±s.e.m. (n=5-7 mice per group). *, p<0.05 vs. vehicle control.

FIGS. 3A1 and 3A2 show vagus nerve stimulation requires a7nAChR to increase platelet cytosolic calcium, enhance cellular activation, and accelerate local clot formation. In FIG. 3A, C57BL6/J mice received vagus nerve stimulation or sham stimulation before harvesting circulating platelets to measure basal cytosolic calcium uptake. Data are presented as mean±s.e.m. (n=5-6 mice per group). **, p<0.01 vs. sham. FIG. 3A2 shows a whisker plot of the data shown in FIG. 3A.

FIG. 3B1, a7nAChR-deficient mice received vagus nerve stimulation or sham stimulation before harvesting circulating platelets to measure basal cytosolic calcium uptake. Data are presented as mean±s.e.m. (n=4 mice per group). p=NS vs. sham. FIG. 3B2 shows a whisker plot of the data shown in FIG. 3BA.

FIG. 3C, C57BL6/J mice received vagus nerve stimulation or sham stimulation before harvesting platelets for analysis of P-selectin (CD62P) expression. Data are presented as mean±s.e.m. (n=12-13 mice per group). p=NS vs. sham.

FIG. 3D, Representative FACS histogram of platelet P-selectin (CD62P) expression after vagus nerve stimulation or sham stimulation.

FIG. 3E, C57BL6/J mice received vagus nerve stimulation or sham stimulation before harvesting platelets, ex vivo thrombin stimulation, and analysis for P-selectin (CD62P) expression. Data are presented as mean±s.e.m. (n=10-12 mice per group). *, p<0.05 vs. sham.

FIG. 3F, Representative FACS histogram of platelet P-selectin (CD62P) expression after vagus nerve stimulation or sham stimulation followed by ex vivo thrombin stimulation.

FIG. 3G, a7nAChR-deficient mice received vagus nerve stimulation or sham stimulation before harvesting platelets, ex vivo thrombin stimulation, and analysis for P-selectin (CD62P) expression. Data are presented as mean±s.e.m. (n=4 mice per group). *, p<0.05 vs. sham.

FIG. 3H, C57BL6/J mice received vagus nerve stimulation or sham stimulation before tail transection and collection of circulating and local shed blood for measurement of (TAT) complexes. Data are presented as mean±s.e.m. (n=3-4 mice per group). **, p<0.01 vs. sham.

FIG. 4 Illustrates how vagus nerve stimulation harnesses acetylcholine-producing ChAT+ T cells in spleen to stimulate platelet calcium uptake via an α7nAChR-dependent mechanism. Vagus nerve-primed platelets travel to sites of tissue injury, enhance thrombin production, accelerate thrombosis, and reduce bleeding.

FIG. 5A Effect of vagus nerve stimulation on platelet a7nAChR expression. a, C57BL6/J mice received vagus nerve stimulation or sham stimulation before harvesting circulating platelets to measure percent expression of a7nAChR. Data are presented as mean±s.e.m. (n=8-9 mice per group). p=NS vs. sham. FIG. 5B, C57BL6/J mice received vagus nerve stimulation or sham stimulation before harvesting platelets to measure a7nAChR expression levels. Data are presented as mean±s.e.m. (n=12-14 mice per group). p=NS vs. sham.

FIGS. 6A and 6B show vagus nerve stimulation requires extracellular source to increase platelet calcium. C57BL6/J mice received vagus nerve stimulation or sham stimulation before harvesting circulating platelets to measure basal cytosolic calcium uptake in the absence of extracellular calcium. Data are presented as mean±s.e.m. (n=3 mice per group). p=NS vs. sham. FIG. 6B shows a whisker plot of the data shown in FIG. 6A.

FIGS. 7A-7J show vagus nerve stimulation does not change platelet activated GPIIb/IIIa or phosphatidylserine expression. FIG. 7A shows C57BL6/J mice received vagus nerve stimulation or sham stimulation before harvesting platelets for analysis of activated GPIIb/IIIa (JON/A) expression. Data are presented as mean #s.e.m. (n=13 mice per group). p=NS vs. sham. FIG. 7B shows C57BL6/J mice received vagus nerve stimulation or sham stimulation before harvesting platelets and analysis of phosphatidylserine. Data are presented as mean±s.e.m. (n=13-14 mice per group). p=NS vs. sham. FIG. 7C, C57BL6/J mice received vagus nerve stimulation or sham stimulation before harvesting platelets, ex vivo thrombin stimulation, and analysis of activated GPIIb/IIIa (JON/A) expression. Data are presented as mean±s.e.m. (n=9-10 mice per group). p=NS vs. sham. FIG. 7D, C57BL6/J mice received vagus nerve stimulation or sham stimulation before harvesting platelets, ex vivo thrombin stimulation, and analysis of phosphatidylserine expression. Data are presented as mean±s.e.m. (n=10-11 mice per group). p=NS vs. sham. FIG. 7E, C57BL6/J mice received vagus nerve stimulation or sham stimulation before harvesting platelets, ex vivo collagen stimulation, and analysis for P-selectin (CD62P) expression. Data are presented as mean±s.e.m. (n=7 mice per group). p=NS vs. sham. FIG. 7F, C57BL6/J mice received vagus nerve stimulation or sham stimulation before harvesting platelets, ex vivo ADP stimulation, and analysis for P-selectin (CD62P) expression. Data are presented as mean±s.e.m. (n=10-11 mice per group). p=NS vs. sham. FIG. 7G, C57BL6/J mice received vagus nerve stimulation or sham stimulation before harvesting platelets, ex vivo collagen stimulation, and analysis of activated GPIIb/IIIa (JON/A) expression. Data are presented as mean±s.e.m. (n=8-11 mice per group). p=NS vs. sham. FIG. 7H, C57BL6/J mice received vagus nerve stimulation or sham stimulation before harvesting platelets, ex vivo collagen stimulation, and analysis of phosphatidylserine expression. Data are presented as mean±s.e.m. (n=8 mice per group). p=NS vs. sham. FIG. 7I, C57BL6/J mice received vagus nerve stimulation or sham stimulation before harvesting platelets, ex vivo ADP stimulation, and analysis of activated GPIIb/IIIa (JON/A) expression. Data are presented as mean±s.e.m. (n=10 mice per group). p=NS vs. sham. FIG. 7J, C57BL6/J mice received vagus nerve stimulation or sham stimulation before harvesting platelets, ex vivo ADP stimulation, and analysis of phosphatidylserine expression. Data are presented as mean±s.e.m. (n=7 mice per group). p=NS vs. sham.

FIGS. 8A-8F show vagus nerve stimulation does not change heart rate, systemic blood pressure, or regional blood flow. FIG. 8A, C57BL6/J mice underwent baseline heart rate measurement before and five minutes after vagus nerve stimulation. Data are presented as mean±s.e.m. Pre-VNS (n=6) and Post-VNS (n=6) mice per group. p=0.15 vs. baseline. Statistical significance was determined by unpaired two-tailed Student's t-test. Figure represents pooled results from 2 experiments performed independently. FIG. 8B, C57BL6/J mice underwent baseline mean arterial blood pressure measurement before and five minutes after vagus nerve stimulation. Data are presented as mean±s.e.m. Pre-VNS (n=6) and Post-VNS (n=6) mice per group. p=0.73 vs. baseline. Statistical significance was determined by unpaired two-tailed Student's t-test. Figure represents pooled results from 2 experiments performed independently. FIG. 8C, α7nAChR-deficient (α7KO) mice underwent baseline heart rate measurement before and five minutes after vagus nerve stimulation. Data are presented as mean #s.e.m. Pre-VNS (n=5) and Post-VNS (n=4) mice per group. p=0.77 vs. baseline. Statistical significance was determined by unpaired two-tailed Student's t-test. Figure represents pooled results from 2 experiments performed independently. FIG. 8D, α7nAChR-deficient (α7KO) mice underwent baseline mean arterial blood pressure measurement before and five minutes after vagus nerve stimulation. Data are presented as mean #s.e.m. Pre-VNS (n=5) and Post-VNS (n=4) mice per group. p=0.46 vs. baseline. Statistical significance was determined by unpaired two-tailed Student's t-test. Figure represents pooled results from 2 experiments performed independently.

FIG. 8E, Athymic nude (Foxn1nu) mice underwent baseline heart rate measurement before and five minutes after vagus nerve stimulation. Data are presented as mean±s.e.m. Pre-VNS (n=4) and Post-VNS (n=4) mice per group. p=0.44 vs. baseline. Statistical significance was determined by unpaired two-tailed Student's t-test. Figure represents pooled results from 2 experiments performed independently. FIG. 8F, athymic nude (Foxn1nu) mice underwent baseline mean arterial blood pressure measurement before and five minutes after vagus nerve stimulation. Data are presented as mean±s.e.m. Pre-VNS (n=4) and Post-VNS (n=4) mice per group. p=0.85 vs. baseline. Statistical significance was determined by unpaired two-tailed Student's t-test. Figure represents pooled results from 2 experiments performed independently. FIG. 8G, C57BL6/J mice underwent baseline tail blood flow measurement before and five minutes after vagus nerve stimulation. Data are presented as mean±s.e.m. Pre-VNS (n=6) and Post-VNS (n=6) mice per group. p=0.29 vs. baseline. Statistical significance was determined by unpaired two-tailed Student's t-test. Figure represents pooled results from 2 experiments performed independently. FIG. 8H, C57BL6/J mice underwent baseline tail blood volume measurement before and five minutes after vagus nerve stimulation. Data are presented as mean±s.e.m. Pre-VNS (n=6) and Post-VNS (n=6) mice per group. p=0.57 vs. baseline. Statistical significance was determined by unpaired two-tailed Student's t-test. Figure represents pooled results from 2 experiments performed independently. Source data are provided as a Source Data File.

FIGS. 9A-9B show platelet gating strategy for flow cytometry. FIG. 9A, Preliminary FSC/SSC gates for starting platelet cell population from murine systemic whole blood. FIG. 9B, Platelet cell population identification with anti-CD41a.

FIG. 10 and FIG. 11 illustrate additional experiments performed using flow cytometry involving leukocytes that express CD41, a platelet marker, and CD4, a T-cell marker, in spleen and blood. The results are summarized in FIGS. 10 and 11. FIG. 10 depicts the percent of total leukocytes that express CD41⁺/CD4⁺ in spleen. FIG. 7 depicts the percent of total leukocytes that express CD41⁺/CD4⁺ in blood. These results indicate that platelets and CD4⁺ cells are in contact with each other. Additionally, immunohistochemistry was performed on ChAT-TdTomato mouse spleens to show the relationship of CD4⁺ChAT⁺ T lymphocytes with circulating platelets and confocal microscopic images are presented in FIGS. 2d and 2e of Bravo-Iniguez et al. Nat Commun (2023) 14:3122, herein incorporated by reference. FIG. 2d of Bravo-Iniguez et al is a representative confocal microscopy image of spleen from ChAT-TdTomato mouse with immunostaining of CD41⁺ platelets (green), CD4+ T lymphocytes (purple), and ChAT-eGFP+ T lymphocytes (red) throughout spleen but mostly in the peripheral white pulp (arrows). FIG. 2e Bravo-Iniguez et al is a representative merged confocal microscopy image of spleen from ChAT-TdTomato mouse showing CD4⁺ChAT-eGFP⁺ T lymphocyte (red and pink, center) in direct contact with CD41⁺ platelets (green). Together these results support the conclusion that vagus nerve stimulation stimulates T cells to release acetylcholine and signal platelets via platelet alpha 7 nicotinic acetylcholine receptors (α7nAChR).

### DETAILED DESCRIPTION

Primary hemostasis after blood vessel injury induces local vasoconstriction and recruitment of circulating platelets to form an initial platelet plug. Secondary hemostasis occurs on the surface of platelets where the tissue factor and contact activation pathways generate thrombin (coagulation factor II), which converts soluble fibrin into insoluble fibrin to stabilize the developing clot. Hemophilia A results from a congenital or acquired impairment in coagulation factor VIII activity, most often caused by genetic mutations that reduce or eliminate factor VIII production. Patients with hemophilia generate insufficient quantities of thrombin and fibrin, resulting in clot instability. The clinical hallmarks are hemorrhage after injury and spontaneous bleeding into muscles and joints. Because the incidence of bleeding complications correlates with circulating factor VIII levels, traditional and current therapies are based on replacing factor VIII through exogenous infusions. Administration of recombinant factor VIII concentrate mitigates transmission of hepatitis or human immunodeficiency virus. However, more than 30% of children with severe hemophilia A cease responding to therapy because they develop neutralizing (inhibitor) factor VIII antibodies. Treatment then shifts to prothrombin complex concentrates or recombinant factor VIIa to bypass factor VIII, but these alternative thrombotic agents are very expensive and associated with significant morbidity and mortality.

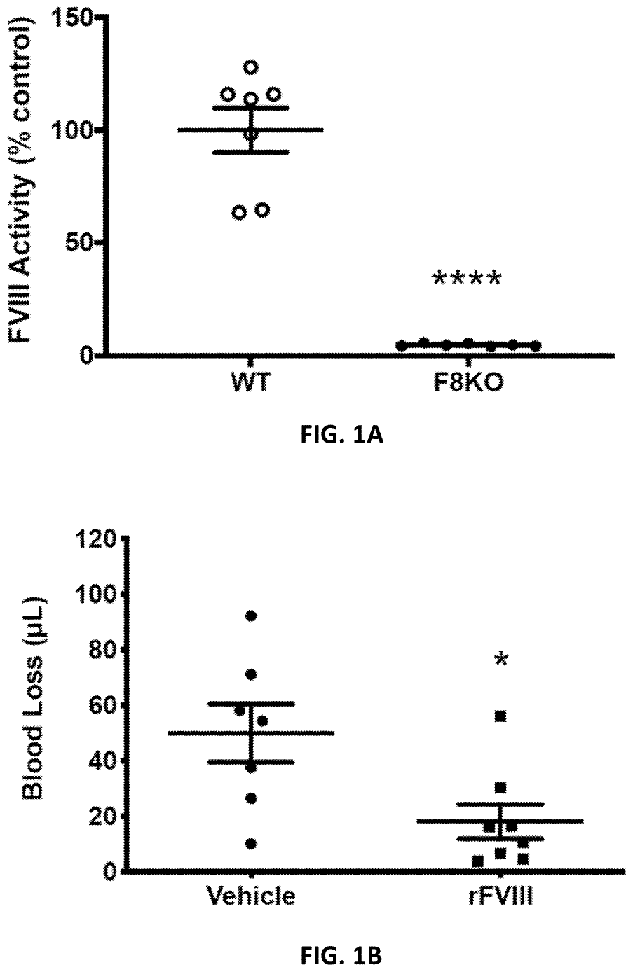

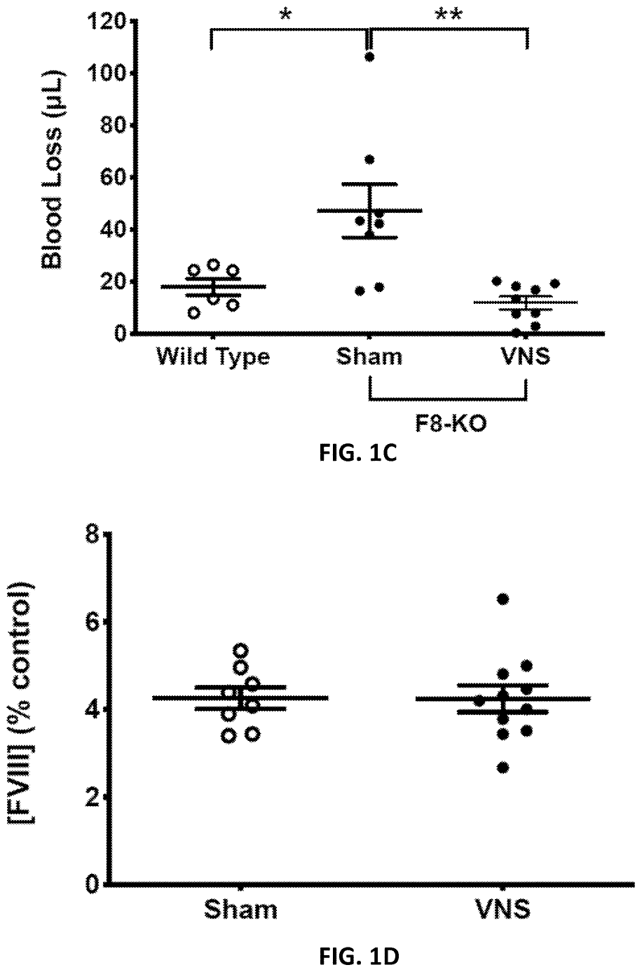

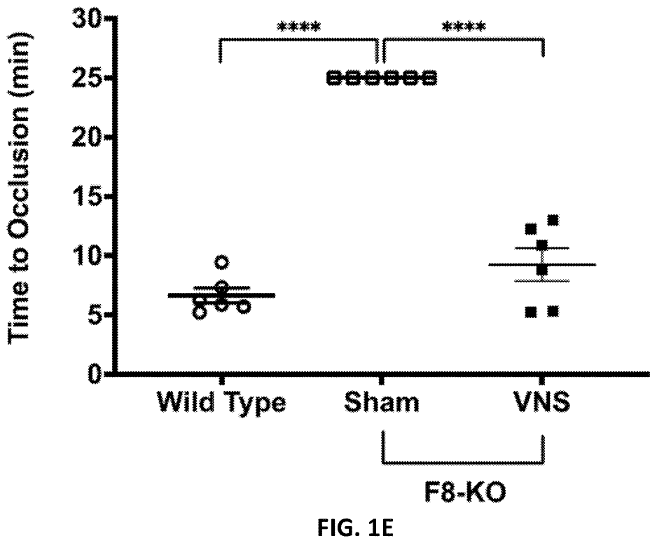

The autonomic nervous system regulates tissue perfusion and hemostasis. Norepinephrine release from perivascular sympathetic neurons mediates arteriolar vasoconstriction to decrease blood flow and facilitate clot formation after injury. Preganglionic sympathetic neurons stimulate the adrenal medulla to secrete epinephrine that activates circulating platelets, the body's primary effector cells to support clotting. Parasympathetic activation mediated by the vagus nerve can support clot formation following traumatic hemorrhage by slowing heart rate to reduce blood pressure. Preclinical evidence shows that electrical vagus nerve stimulation significantly reduces blood loss and duration of bleeding after soft tissue injury. Vagus nerve stimulation accelerates clot initiation as measured by rotational thromboelastography (RoTEG), a viscoelastic technique to quantify interactions of plasma coagulation factors and inhibitors with blood cells. Vagus nerve stimulation increases early thrombin generation at the injury site without significantly changing circulating thrombin levels. Accordingly, here we reasoned vagus nerve stimulation might enhance clot formation and reduce bleeding in hemophilia A. To study vagus nerve stimulation in hemophilia A, we utilized factor VIII-knockout (F8KO) mice derived via targeted mutation of the factor VIII gene resulting in defective protein production. To validate the bleeding model, we measured circulating factor VIII activity in F8KO mice and found it is significantly reduced compared with wild-type control animals (FIG. 1a). We then administered recombinant factor VIII, a clinical hemophilia A therapy, to F8KO mice before tail transection causing uncontrolled arterial hemorrhage, and recorded a significant 64% reduction in blood loss as compared with vehicle treatment (FIG. 1b). After administering five minutes of vagus nerve stimulation to the F8KO mice, we observed a significant 75% reduction in blood loss as compared with sham stimulation (FIG. 1c). Because the vagus nerve innervates liver which produces factor VIII, we wondered if vagus nerve stimulation stimulates factor VIII synthesis. Our results show vagus nerve stimulation does not increase systemic factor VIII activity as compared with sham stimulation (FIG. 1d). To confirm vagus nerve stimulation accelerates clotting, we utilized a carotid artery endothelial injury model that quantifies platelet activation and thrombus formation. We observed that vagus nerve stimulation in F8KO mice significantly reduces time to carotid occlusion after injury as compared with sham stimulation. By analyzing lung sections for signs of systemic hypercoagulability after vagus nerve stimulation, we observed normal tissue architecture without evidence of arterial clots, alveolar congestion or hemorrhage associated with thromboembolism (FIG. 1E). Together these findings show vagus nerve stimulation reduces blood loss and accelerates clot formation in hemophilia A mice without increasing circulating factor VIII activity or inducing pulmonary thrombosis.

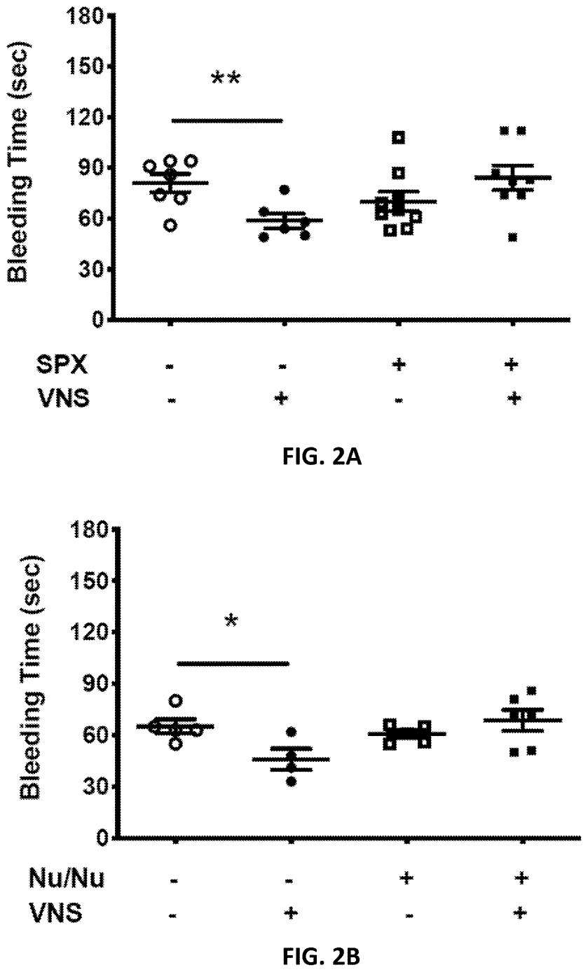

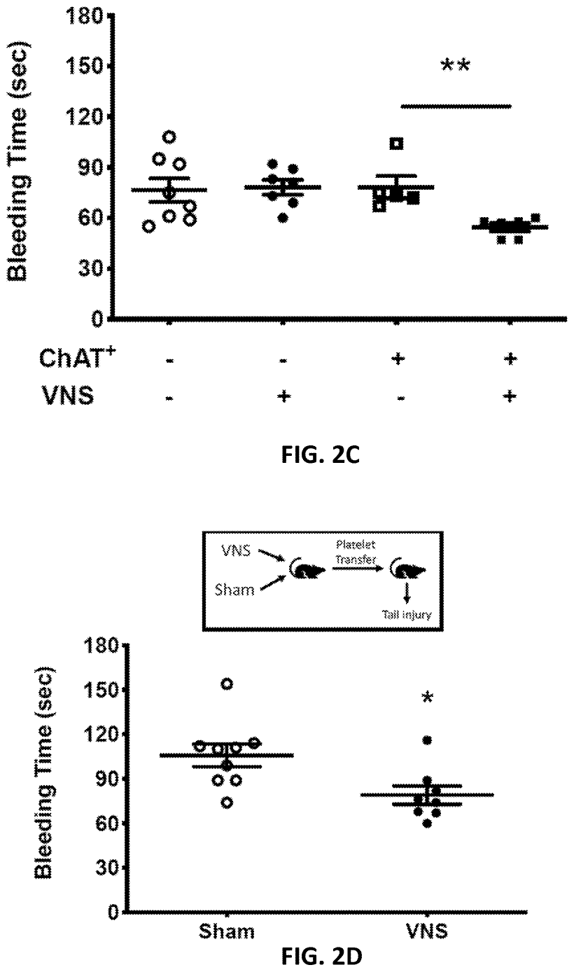

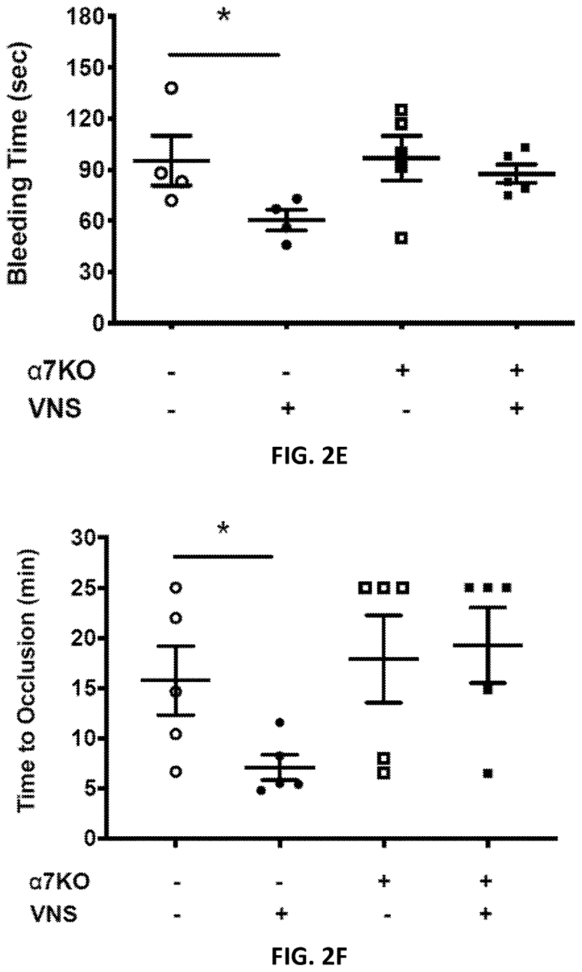

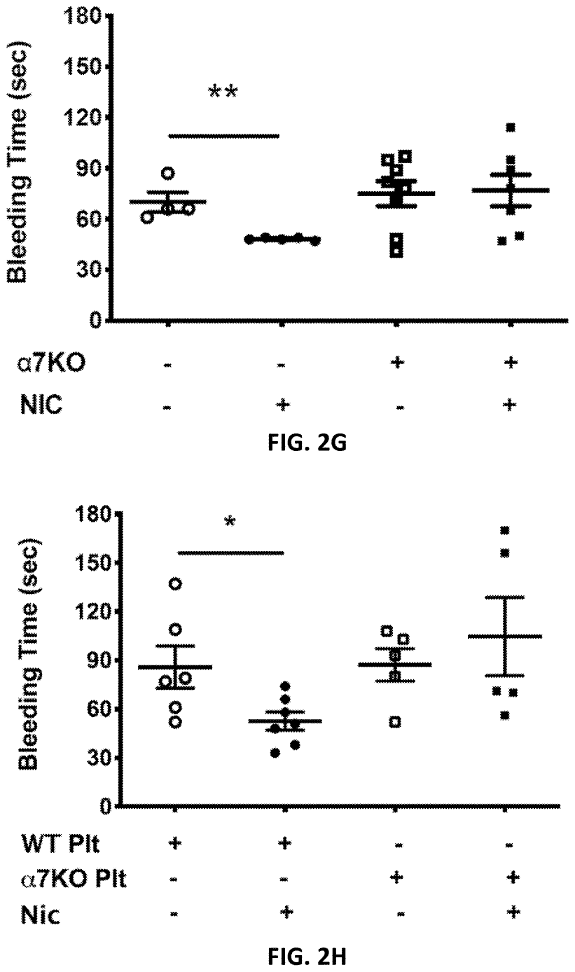

Previous studies demonstrate vagus nerve stimulation inhibits neutrophil migration to sites of peripheral inflammation and decreases expression of CD11b, a beta (2)-integrin important to cellular adhesion and chemotaxis, but only in the presence of an intact spleen. Because the mouse tail is not innervated by the vagus nerve, we reasoned vagus nerve stimulation facilitates local thrombosis to reduce bleeding by influencing circulating platelets in spleen. Accordingly, we performed splenectomy before tail transection. While we did not observe a difference in bleeding times between splenectomized animals and sham-splenectomized controls, vagus nerve stimulation fails to decrease bleeding time in splenectomized mice as compared with sham stimulation (FIG. 2a).

Prior evidence establishes that vagus nerve stimulation induces a distinct subset of memory T cells (CD4+ CD44high CD62low ChAT-EGFP+) expressing choline acetyltransferase, the enzyme that catalyzes acetylcholine synthesis, to increase acetylcholine levels in spleen and inhibit cytokine production by local tissue macrophages. To determine the role of ChAT-EGFP+ T lymphocytes in hemostasis, we first performed tail transections in athymic nude (Foxn1nu) mice lacking mature functional lymphocytes. We did not observe a difference in bleeding time between Foxn1nu mice and wild-type mice (FIG. 2b). Vagus nerve stimulation fails to decrease bleeding time in Foxn1nu mice as compared with sham stimulation (FIG. 2b). We then isolated ChAT-EGFP+ or control ChAT-EGFP-splenic cells from ChAT (BAC)-EGFP mice and transferred them into Foxn1nu mice before tail injury. Vagus nerve stimulation significantly reduces bleeding time in Foxn1nu mice reconstituted with ChAT-EGFP+ T lymphocytes, but fails to shorten bleeding time in animals given ChAT-EGFP-T cells (FIG. 2c). Collectively these findings indicate vagus nerve stimulation reduces bleeding time through a mechanism that requires splenic ChAT-EGFP+ T lymphocytes.

Acetylcholine and other cholinergic agonists enhance human platelet activation via a7 nicotinic acetylcholine receptors (a7nAChR). Considering one-third of circulating platelets reside in spleen, we reasoned splenic ChAT-EGFP+ T-cell acetylcholine secretion controls platelet a7nAChR function. To explore this, we performed vagus nerve stimulation on uninjured wild-type mice and transferred circulating platelets into naïve animals undergoing tail transection. Platelets from vagus nerve stimulated mice significantly reduce bleeding time in recipient animals as compared with platelets from sham-stimulated mice (FIG. 2D). We performed complete blood counts on the donor mice to determine if platelet number changes after vagus nerve stimulation. There is no significant difference in platelet count or other circulating blood cell counts after vagus nerve stimulation as compared with sham stimulation (Table 1). Next, we studied hemostasis in a7nAChR-deficient (a7KO) mice. We did not observe a difference in bleeding time between a7KO and wild-type littermate mice (FIG. 2E). Vagus nerve stimulation significantly reduces tail bleeding time in wild-type mice as compared with sham stimulation, but fails to reduce bleeding time in a7KO mice (FIG. 2E). After carotid artery injury, we did not observe a difference in occlusion time between a7KO and wild-type mice (FIG. 2F). Vagus nerve stimulation significantly reduces time to carotid occlusion in wild-type animals as compared with sham stimulation, but fails to decrease occlusion time in a7KO mice (FIG. 2F). Together these findings suggest vagus nerve stimulation accelerates local thrombosis to decrease bleeding through a mechanism requiring a7nAChR.

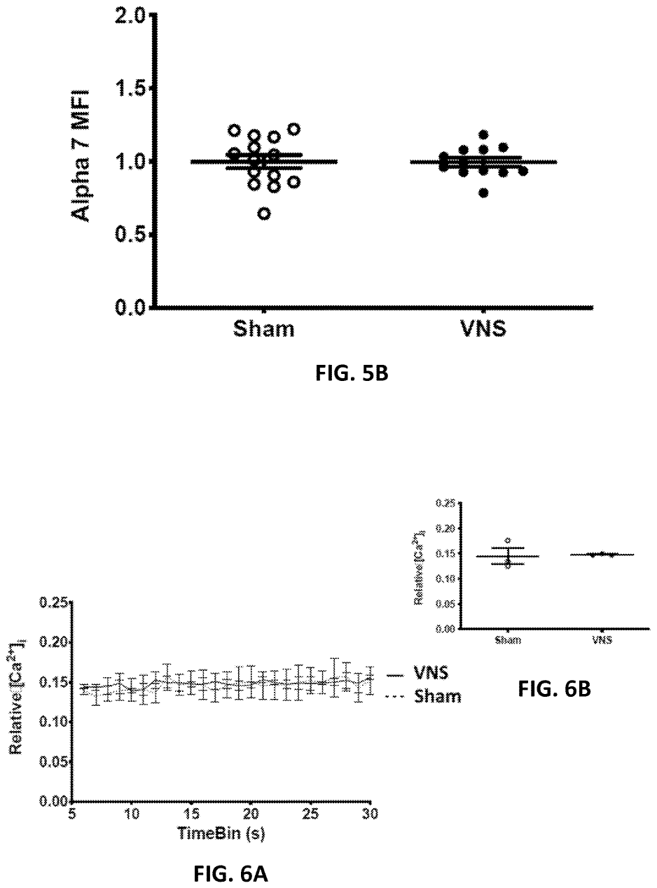

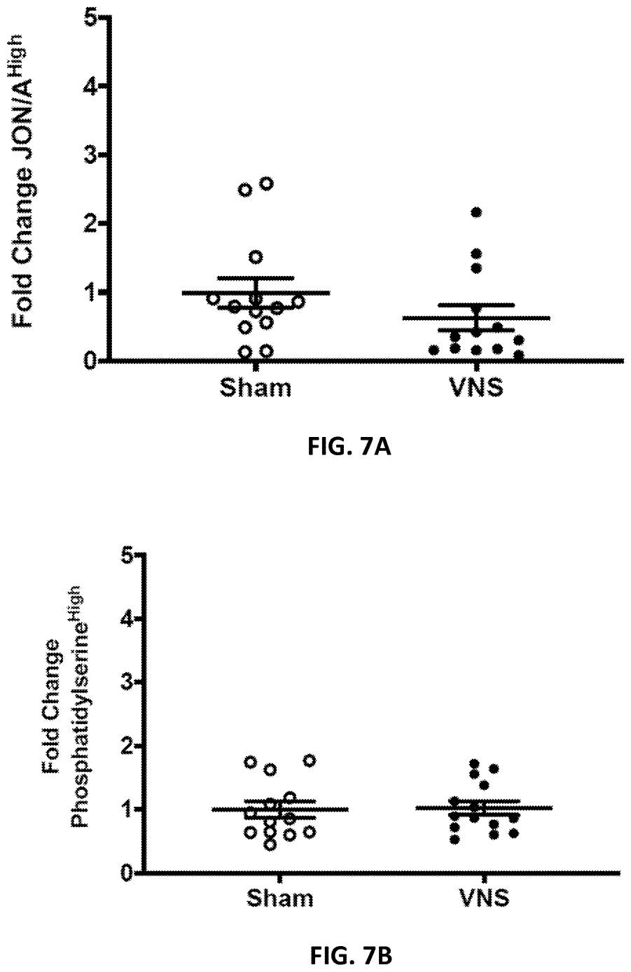

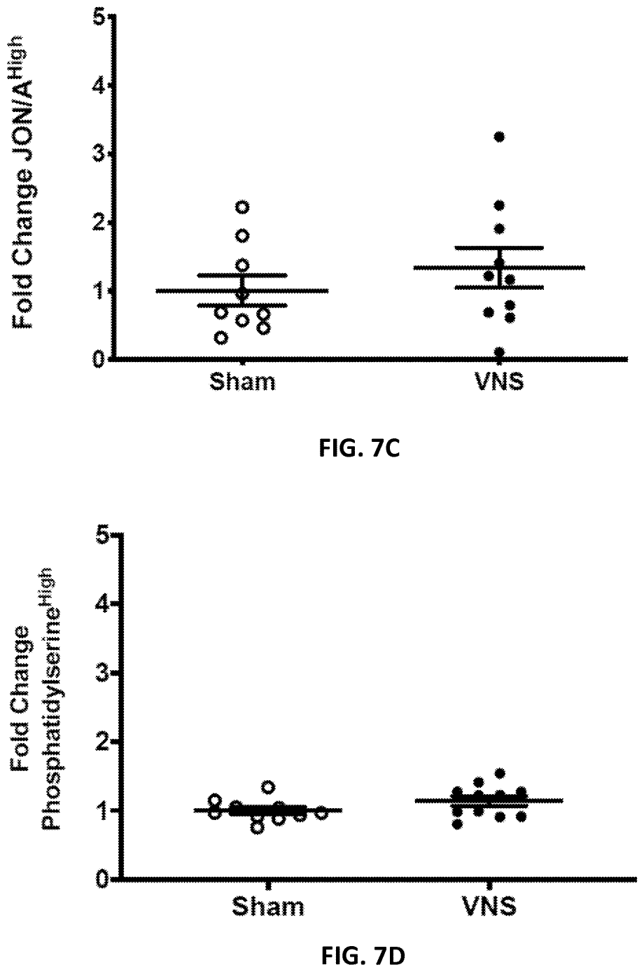

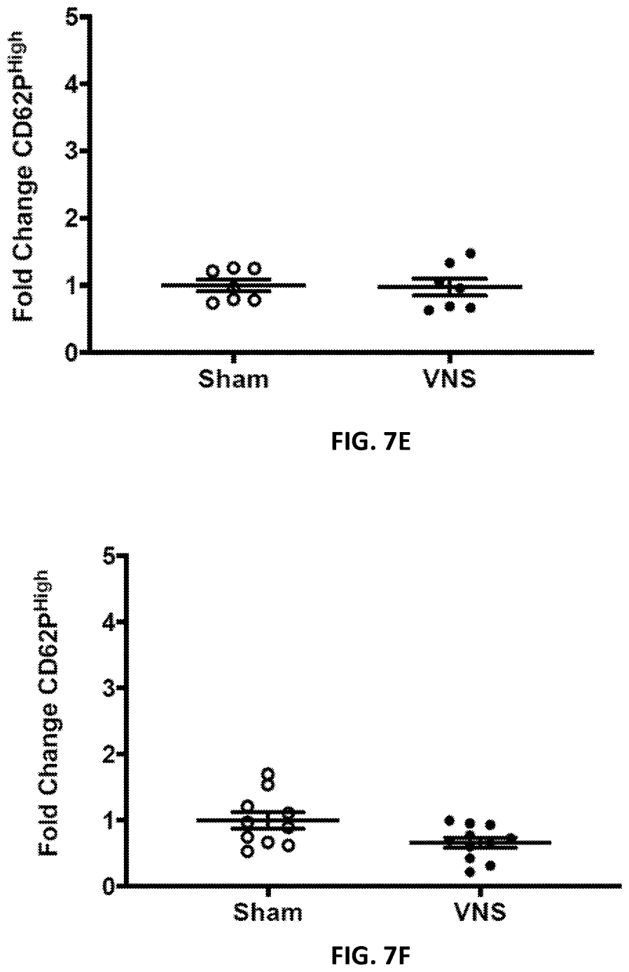

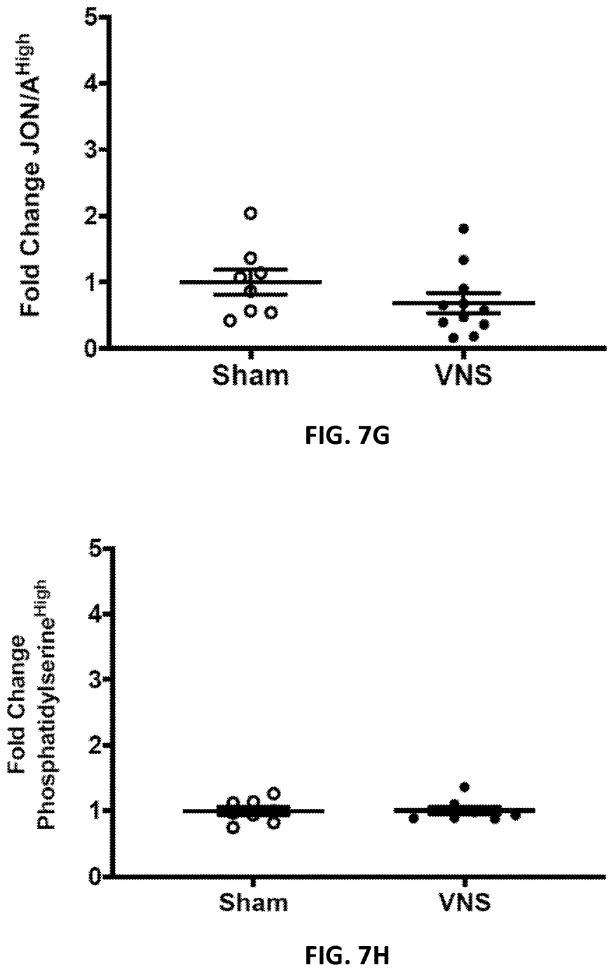

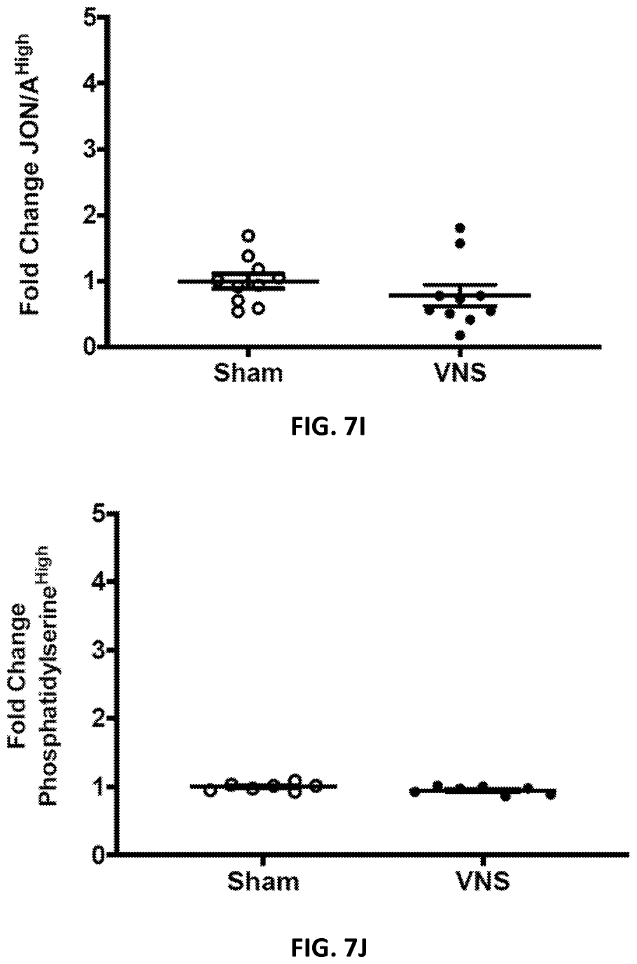

To confirm platelet a7nAChR modulates hemostasis, we first administered the pharmacological a7nAChR agonist nicotine to wild-type mice before tail injury. Nicotine significantly decreases bleeding time as compared with vehicle treatment (FIG. 2G). Similar to vagus nerve stimulation, nicotine fails to reduce bleeding time in a7nAChR-deficient mice (FIG. 2G). To restrict a7nAChR to platelets, we transferred platelets from wild-type or a7KO donor mice into a7nAChR-deficient animals before tail transection. Nicotine significantly reduces bleeding time in a7 a7nAChR-deficient mice reconstituted with wild-type platelets, but not in animals receiving platelets from a7KO donors (FIG. 2H). To evaluate platelet a7nAChR function, FACS analysis reveals similar percentages of platelets expressing a7nAChR after vagus nerve stimulation or sham stimulation (FIG. 5A). We did not observe significant differences in a7nAChR expression levels on platelets after vagus nerve stimulation as compared with sham stimulation (FIG. 5B). Previous in vitro experiments demonstrate a7nAChR modulates platelet calcium uptake and intracellular calcium governs essential platelet activation pathways. By quantifying platelet cytosolic calcium levels, we found vagus nerve stimulation significantly increases basal intracellular calcium concentrations as compared with sham stimulation (FIG. 7A). Vagus nerve stimulation fails to increase platelet cytosolic calcium in a7nAChR-deficient mice (FIG. 5B), or when the extracellular calcium ion source is removed (FIG. 6A). Collectively these findings suggest vagus nerve stimulation harnesses acetylcholine release from ChAT-EGFP+ T cells to increase platelet cytosolic calcium via a7nAChR.

Platelet activation at an injury site is characterized by fusion of intracellular alpha granules with the plasma membrane to release prothrombotic proteins including P-selectin, conformational change to the active primary fibrinogen receptor GPIIb/IIIa that facilitates cross-linking and aggregation, and increased expression of membrane phosphatidylserine that promotes prothrombinase complex formation and clot formation. To explore these cellular activation events, we first analyzed circulating platelets from healthy uninjured mice administered vagus nerve stimulation. Compared with sham stimulation, platelets remain quiescent after vagus nerve stimulation without increased expression of P-selectin (FIGS. 7C and 7D, representative image), the active GPIIb/IIIa receptor (FIG. 7A), or phosphatidylserine (FIG. 7B).

We then performed vagus nerve stimulation and exposed harvested circulating platelets to the calcium-dependent agonists thrombin, collagen, or adenosine diphosphate (ADP). In the presence of thrombin, significantly more platelets express P-selectin after vagus nerve stimulation as compared with sham stimulation (FIGS. 7E-7F, representative image). In contrast, expression of active GPIIb/IIIa or phosphatidylserine remains unchanged after vagus nerve stimulation as compared with sham stimulation (FIG. 7C-D). In a7nAChR-deficient mice, vagus nerve stimulation fails to increase P-selectin expression after thrombin exposure as compared with sham stimulation (FIG. 7G), further supporting the essential role of a7nAChR to platelet activation. In the presence of collagen or ADP, we did not observe any difference in P-selectin expression between vagus nerve stimulation or sham stimulation (FIGS. 7E-F). We did not observe any difference in active GPIIb/IIIa receptor or phosphatidylserine expression between vagus nerve stimulation or sham stimulation following exposure to collagen (FIGS. 7G-7H) or ADP (FIGS. 7I-7J). Together these results suggest vagus nerve stimulation enhances platelet activation by selectively increasing thrombin-mediated expression of P-selectin.

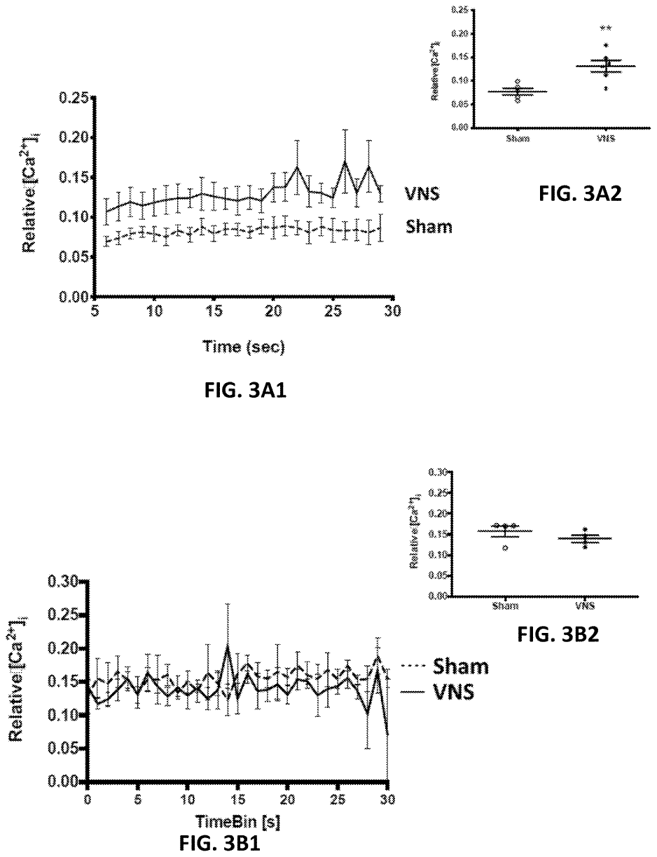

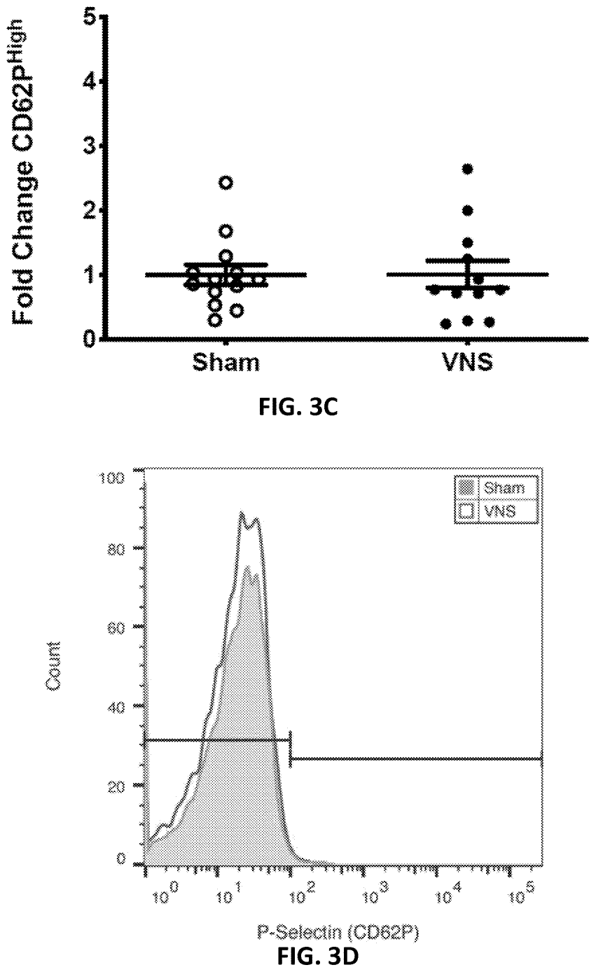

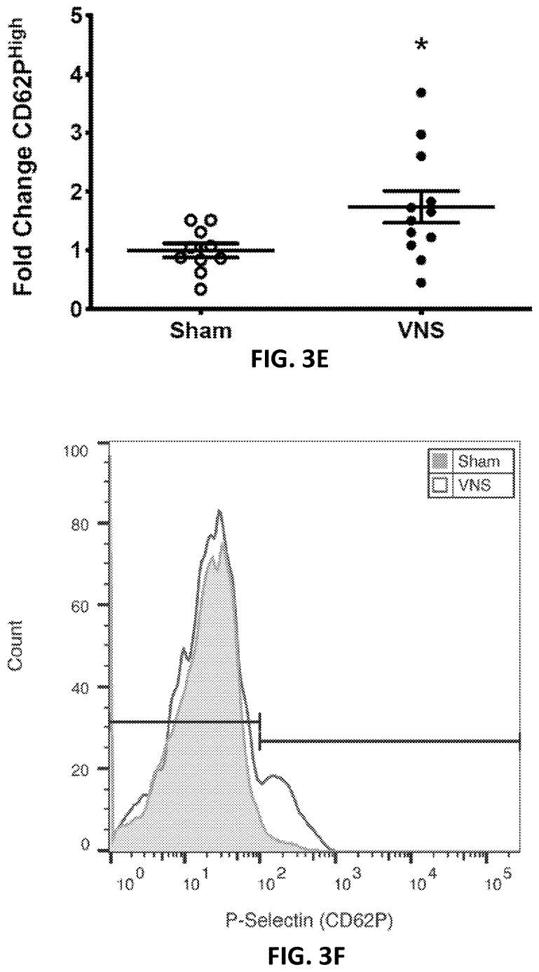

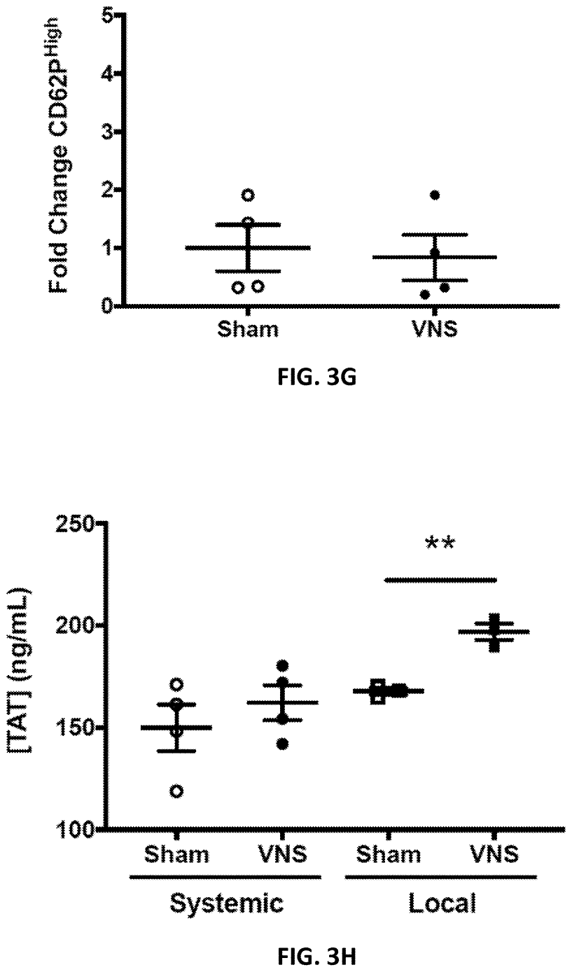

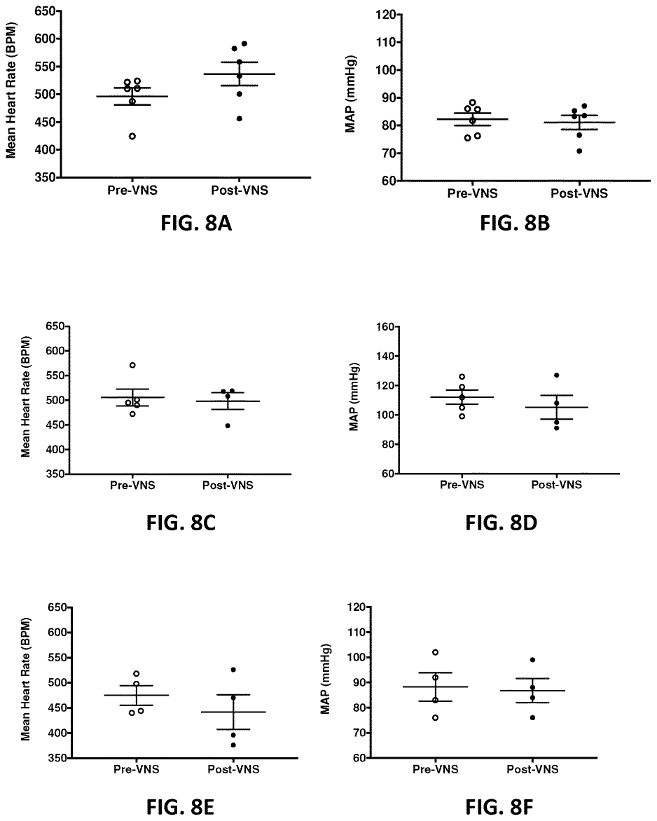

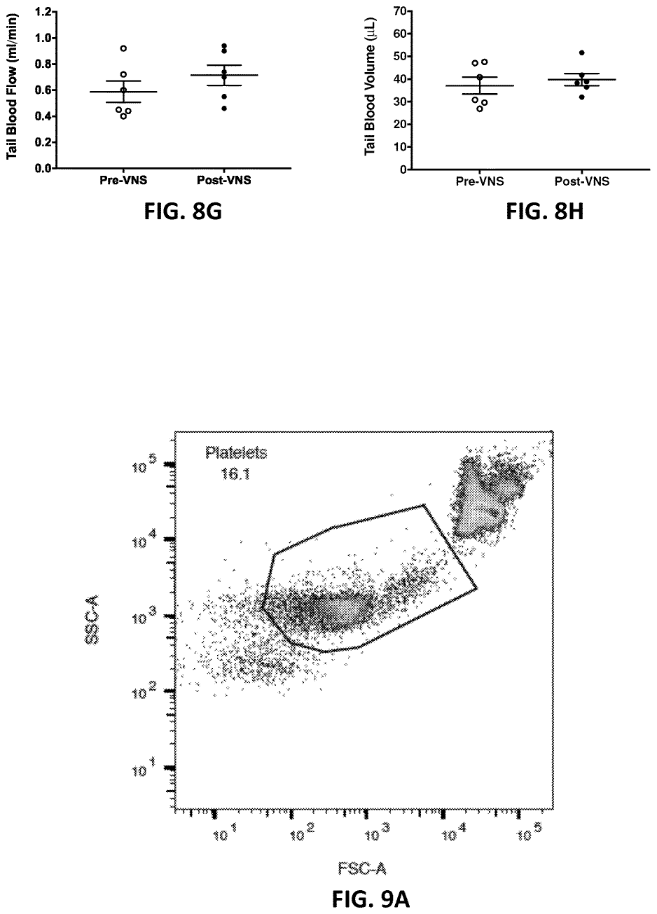

Recruitment and activation of circulating platelets at a blood vessel injury site supports rapid thrombin generation that is crucial for fibrin deposition and stabilization of the developing clot. We evaluated thrombin production by measuring thrombin-antithrombin (TAT) complex concentrations. Vagus nerve stimulation significantly increases local TAT complex levels at the tail transection site as compared with sham stimulation (FIG. 3h). We did not observe any significant differences in systemic TAT complex levels between vagus nerve stimulation and sham stimulation (FIG. 3h). Similar to hemophilia A mice, wild-type animals demonstrate normal pulmonary architecture without evidence of thromboembolism after vagus nerve stimulation. Finally, to determine if parasympathetic to determine if the parasympathetic effects of vagus nerve stimulation contribute to hemostasis, we performed electrocardiograms and measured systemic blood pressure in wild-type, α7KO, and Foxn1nu mice. Compared with pre-stimulation, we did not observe any differences in post-stimulation mean heart rate or arterial blood pressure in wild-type (FIGS. 8A-8B), α7KO (FIGS. 8C-8D), or Foxn1nu mice (FIGS. 8E-8F). Furthermore, we did not observe any differences in post-stimulation tail blood flow (FIG. 8G) or tail blood volume in wild-type mice (FIG. 8H). These findings suggest vagus nerve stimulation primes circulating platelets to accelerate local clot formation without inducing systemic thrombosis or influencing systemic hemodynamics or tissue perfusion to the site of injury.

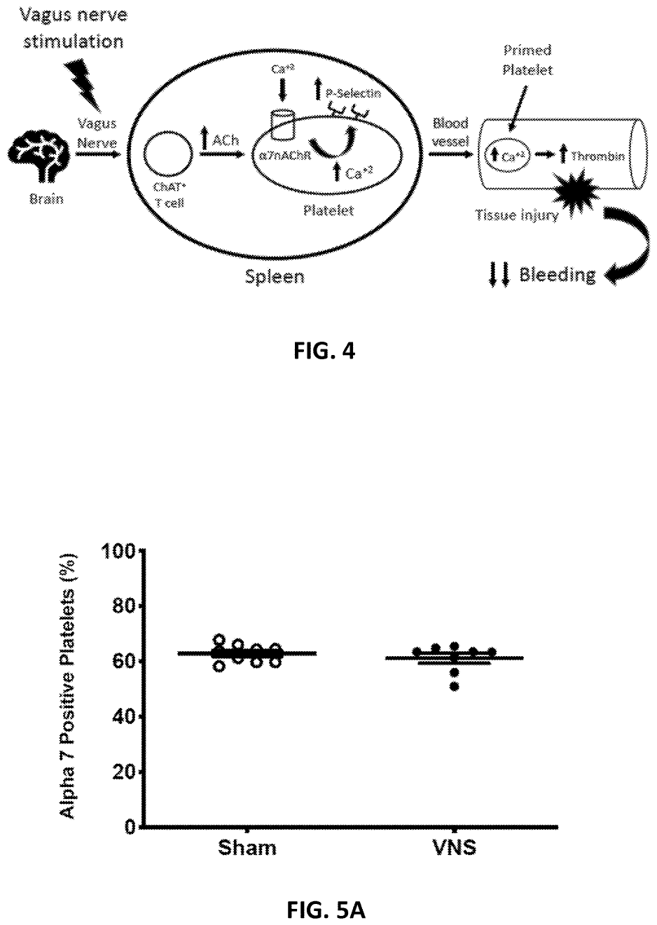

Taken together, these studies reveal a previously unrecognized neural hemostatic mechanism (FIG. 4). The vagus nerve targets spleen to access circulating platelets that occupy a critical role in hemostasis. Previously implicated in relaying neural signals in the inflammatory reflex, regulating blood pressure and vascular contractility via endothelial cholinergic receptors, and controlling cytotoxic T cells and clearance of chronic viral infection, the data here implicate memory subtype (CD4+ CD44high CD62low ChAT-EGFP+) T lymphocytes in regulation of platelet function and hemostasis. Following vagus nerve stimulation, ChAT-EGFP+ T cell release of acetylcholine may potentiate platelet a7nAChR signaling in spleen by avoiding rapid degradation via circulating blood acetylcholinesterases. Release of other endogenous ligands following vagus nerve stimulation may contribute to platelet activation. Previous work shows splenic lymphocytes secrete ChAT, which may synthesize additional extracellular acetylcholine to support a7nAChR signaling. Establishing a role for ChAT-EGFP+T lymphocytes in modulating bleeding provides further insights into how hemostatic and immunologic mechanisms control the host response to injury.

Circulating platelets are quiescent unless exposed to vascular injury, where their upregulation of coagulation factor activity creates a stable fibrin clot. Since vagus nerve stimulation increases platelet cytosolic calcium via a7nAChR to facilitate activation, this priming phenomenon enhances systemic platelet function and extends neural control of hemostasis to anatomic sites lacking direct vagal innervation. Vagus nerve stimulation does not facilitate activation in all platelets, which may relate, in part, to only 60% expressing surface a7nAChR. Because we utilized acute hemorrhage and thrombosis models, it is unclear whether platelets remain in a primed state after vagus nerve stimulation or if repeated stimulation is necessary to enhance their function. Ongoing platelet clearance from the circulation may also impact hemostasis during chronic vagus nerve stimulation.

Although a7nAChR functions as a traditional ligand-gated ion channel in neuronal cells, it activates G-protein coupled, inositol triphosphate (IP3)-induced calcium release in neuronal and non-neuronal immune cells. Our finding that vagus nerve stimulation requires extracellular calcium to increase platelet cytosolic calcium is consistent with cholinergic signaling in human platelets and suggests canonical a7nAChR ion channels facilitate platelet activation, accelerated thrombosis, and decreased bleeding. Additional studies may determine if metabotropic a7nAChR signaling upregulates platelets after vagus nerve stimulation. Because transferring calcium-primed platelets to naïve animals improves hemostasis, administering vagus nerve stimulation to blood donors may improve platelet function in recipients. Vagus nerve stimulation may reduce overall transfusion volumes, minimize transfusion-related complications and costs, and ease the burden on blood bank stores.

Increasing evidence suggests bioelectronic approaches are feasible for chronic treatment of medically-refractory depression, epilepsy, rheumatoid arthritis, and Crohn's disease. Since 1997, more than 100,000 patients have received vagus nerve stimulation via implantable pulse generators. Despite their favorable safety profile, surgery poses a risk of bleeding, infection, and injury to important anatomic structures. The advent of noninvasive approaches, such as transcutaneous cervical vagus nerve stimulation and transcutaneous stimulation of the auricular branch of the vagus nerve, may mitigate these risks. Noninvasive techniques may allow for emergent treatment of traumatic injury in individuals not fitted with an implantable device. Hemophilia A affects hundreds of thousands of individuals and causes significant morbidity and mortality. It may be useful to explore the use of vagus nerve stimulation to treat hemophilia A and other bleeding disorders in clinical trials.

TABLE 1
Complete blood cell counts

| | SHAM | | VNS | | |
|---|---|---|---|---|---|
| | Avg. | S.E.M. | Avg. | S.E.M. | p value |
| WBC (K/μL) | 2.67 | 0.70 | 2.94 | 1.24 | p > 0.05 |
| Hb (g/L) | 12.08 | 0.35 | 11.58 | 0.48 | p > 0.05 |
| Het (%) | 44.03 | 0.68 | 42.20 | 1.19 | p > 0.05 |
| Plt (K/μL) | 758 | 90.5 | 724.3 | 90.9 | p > 0.05 |

n = 4 mice per group

FIGS. 1A-1E Vagus nerve stimulation reduces traumatic blood loss and accelerates clot formation in hemophilia A mice. FIG. 1A, Circulating factor VIII activity in wild-type and factor VIII deficient (hemophilia A) mice. Data are presented as mean±s.e.m. (n=7 mice). ****, p<0.0001 vs. control. FIG. 1B, Hemophilia A mice received rFVIII (Advate®, 200 U/kg, r.o.) or vehicle before tail transection. Data are presented as mean±s.e.m. (n=7-8 mice per group). *, p<0.05 vs. sham. FIG. 1C, Hemophilia A mice received vagus nerve stimulation or sham stimulation before tail transection. Data are presented as mean±s.e.m. (n=8-9 mice per group). **, p<0.01 vs. sham. FIG. 1D, Hemophilia A mice received vagus nerve stimulation or sham stimulation before blood collection to determine factor VIII activity. Data are presented as mean±s.e.m. (n=8-11 mice per group). p=NS vs. sham. e, Hemophilia A mice received vagus nerve stimulation or sham stimulation carotid artery injury and thrombosis. Data are presented as mean±s.e.m. (n=6 mice per group). ****, p<0.0001 vs. sham. f, Representative images of lungs from hemophilia A mice after sham stimulation. FIG. 1G, Representative images of lungs from hemophilia A mice after vagus nerve stimulation.

FIGS. 6A-6H Vagus nerve stimulation harnesses choline acetyltransferase-expressing T cells in spleen to stimulate platelets via a7nAChR. FIG. 6A, C57BL6/J mice underwent splenectomy or sham splenectomy followed by vagus nerve stimulation or sham stimulation before tail transection. Data are presented as mean±s.e.m. (n=6-9 mice per group). **, p<0.01 versus sham. FIG. 6B, Wild-type BALB/c or T lymphocyte deficient (Foxn1nu) mice received vagus nerve stimulation or sham stimulation before tail transection. Data are presented as mean #s.e.m. (n=4-6 mice per group). *, p=0.05 vs. sham. FIG. 6C, T lymphocyte deficient (Foxn1nu) mice were reconstituted with ChAT-eGFP+ or ChAT-eGFP-cells followed by vagus nerve stimulation or sham stimulation before tail transection. Data are presented as mean±s.e.m. (n=5-8 mice per group). **, p<0.01 vs. sham. FIG. 6D, Platelets from uninjured C57BL6/J mice receiving vagus nerve stimulation or sham stimulation were transferred into naïve animals before tail transection. Data are presented as mean±s.e.m. (n=8-9 mice per group). *, p<0.05 vs. sham. FIG. 6E, Wild-type or a7nAChR-deficient mice received vagus nerve stimulation or sham stimulation before tail transection. Data are presented as mean±s.e.m. (n=4-5 mice per group). *, p<0.05 vs. sham. FIG. 6F, Wild-type or a7nAChR-deficient mice received vagus nerve stimulation or sham stimulation before carotid artery injury and thrombosis. Data are presented as mean±s.e.m. (n=5 mice per group). *, p<0.05 vs. sham. FIG. 6G, Wild-type or a7nAChR-deficient mice received nicotine or vehicle before tail transection. Data are presented as mean±s.e.m. (n=4-8 mice per group). **, p<0.01 vs. vehicle control. FIG. 6H, a7nAChR deficient mice were reconstituted with platelets from wild-type or a7nAChR-deficient mice followed by treatment with nicotine or vehicle before tail transection. Data are presented as mean±s.e.m. (n=5-7 mice per group). *, p<0.05 vs. vehicle control.

FIGS. 7A-7H Vagus nerve stimulation requires a7nAChR to increase platelet cytosolic calcium, enhance cellular activation, and accelerate local clot formation. FIG. 7A, C57BL6/J mice received vagus nerve stimulation or sham stimulation before harvesting circulating platelets to measure basal cytosolic calcium uptake. Data are presented as mean±s.e.m. (n=5-6 mice per group). **, p<0.01 vs. sham. FIG. 7B, a7nAChR-deficient mice received vagus nerve stimulation or sham stimulation before harvesting circulating platelets to measure basal cytosolic calcium uptake. Data are presented as mean±s.e.m. (n=4 mice per group). p=NS vs. sham. FIG. 7C, C57BL6/J mice received vagus nerve stimulation or sham stimulation before harvesting platelets for analysis of P-selectin (CD62P) expression. Data are presented as mean±s.e.m. (n=12-13 mice per group). p=NS vs. sham. FIG. 7D, Representative FACS histogram of platelet P-selectin (CD62P) expression after vagus nerve stimulation or sham stimulation. FIG. 7E, C57BL6/J mice received vagus nerve stimulation or sham stimulation before harvesting platelets, ex vivo thrombin stimulation, and analysis for P-selectin (CD62P) expression. Data are presented as mean±s.e.m. (n=10-12 mice per group). *, p<0.05 vs. sham. FIG. 7F, Representative FACS histogram of platelet P-selectin (CD62P) expression after vagus nerve stimulation or sham stimulation followed by ex vivo thrombin stimulation. FIG. 7G, a7nAChR-deficient mice received vagus nerve stimulation or sham stimulation before harvesting platelets, ex vivo thrombin stimulation, and analysis for P-selectin (CD62P) expression. Data are presented as mean±s.e.m. (n=4 mice per group). *, p<0.05 vs. sham. FIG. 7H, C57BL6/J mice received vagus nerve stimulation or sham stimulation before tail transection and collection of circulating and local shed blood for measurement of (TAT) complexes. Data are presented as mean±s.e.m. (n=3-4 mice per group). **, p<0.01 vs. sham. FIG. 7I, Representative lung images from C57BL6/J mice after sham stimulation. FIG. 7J, Representative lung images from C57BL6/J mice after vagus nerve stimulation.

FIG. 4 The Neural Tourniquet. Vagus nerve stimulation harnesses acetylcholine-producing ChAT+ T cells in spleen to stimulate platelet calcium uptake via an a7nAChR-dependent mechanism. Vagus nerve-primed platelets travel to sites of tissue injury, stimulate thrombin production and local thrombosis, and reduce bleeding time.

FIGS. 5A and 5B Effect of vagus nerve stimulation on platelet a7nAChR expression. FIG. 5A shows C57BL6/J mice received vagus nerve stimulation or sham stimulation before harvesting circulating platelets to measure percent expression of a7nAChR. Data are presented as mean±s.e.m. (n=8-9 mice per group). p=NS vs. sham. FIG. 5B shows C57BL6/J mice received vagus nerve stimulation or sham stimulation before harvesting platelets to measure a7nAChR expression levels. Data are presented as mean±s.e.m. (n=12-14 mice per group). p=NS vs. sham.

FIGS. 5A-5B show vagus nerve stimulation requires extracellular source to increase platelet calcium. C57BL6/J mice received vagus nerve stimulation or sham stimulation before harvesting circulating platelets to measure basal cytosolic calcium uptake in the absence of extracellular calcium. Data are presented as mean±s.e.m. (n=3 mice per group). p=NS vs. sham.

FIG. 6A-6B illustrate the effect of vagus nerve stimulation on platelet activated GPIIb/IIIa, phosphatidylserine, or P-selectin expression. a, C57BL6/J mice received vagus nerve stimulation or sham stimulation before harvesting platelets for analysis of activated GPIIb/IIIa (JON/A) expression. Data are presented as mean±s.e.m. (n=13 mice per group). p=NS vs. sham. b, C57BL6/J mice received vagus nerve stimulation or sham stimulation before harvesting platelets and analysis of phosphatidylserine. Data are presented as mean±s.e.m. (n=13-14 mice per group). p=NS vs. sham. c, C57BL6/J mice received vagus nerve stimulation or sham stimulation before harvesting platelets, ex vivo thrombin stimulation, and analysis of activated GPIIb/IIIa (JON/A) expression. Data are presented as mean #s.e.m. (n=9-10 mice per group). p=NS vs. sham. d, C57BL6/J mice received vagus nerve stimulation or sham stimulation before harvesting platelets, ex vivo thrombin stimulation, and analysis of phosphatidylserine expression. Data are presented as mean±s.e.m. (n=10-11 mice per group). p=NS vs. sham. e, C57BL6/J mice received vagus nerve stimulation or sham stimulation before harvesting platelets, ex vivo collagen stimulation, and analysis for P-selectin (CD62P) expression. Data are presented as mean±s.e.m. (n=7 mice per group). p=NS vs. sham. f, C57BL6/J mice received vagus nerve stimulation or sham stimulation before harvesting platelets, ex vivo ADP stimulation, and analysis for P-selectin (CD62P) expression. Data are presented as mean±s.e.m. (n=10-11 mice per group). p=NS vs. sham. g, C57BL6/J mice received vagus nerve stimulation or sham stimulation before harvesting platelets, ex vivo collagen stimulation, and analysis of activated GPIIb/IIIa (JON/A) expression. Data are presented as mean±s.e.m. (n=8-11 mice per group). p=NS vs. sham. h, C57BL6/J mice received vagus nerve stimulation or sham stimulation before harvesting platelets, ex vivo collagen stimulation, and analysis of phosphatidylserine expression. Data are presented as mean±s.e.m. (n=8 mice per group). p=NS vs. sham. i, C57BL6/J mice received vagus nerve stimulation or sham stimulation before harvesting platelets, ex vivo ADP stimulation, and analysis of activated GPIIb/IIIa (JON/A) expression. Data are presented as mean±s.e.m. (n=10 mice per group). p=NS vs. sham. j, C57BL6/J mice received vagus nerve stimulation or sham stimulation before harvesting platelets, ex vivo ADP stimulation, and analysis of phosphatidylserine expression. Data are presented as mean±s.e.m. (n=7 mice per group). p=NS vs. sham.

FIGS. 8A-8F show vagus nerve stimulation does not change heart rate. C57BL6/J mice underwent baseline heart rate measurement before and one minute after vagus nerve stimulation. Data are presented as mean±s.e.m. (n=4 mice per group). p=NS vs. sham.

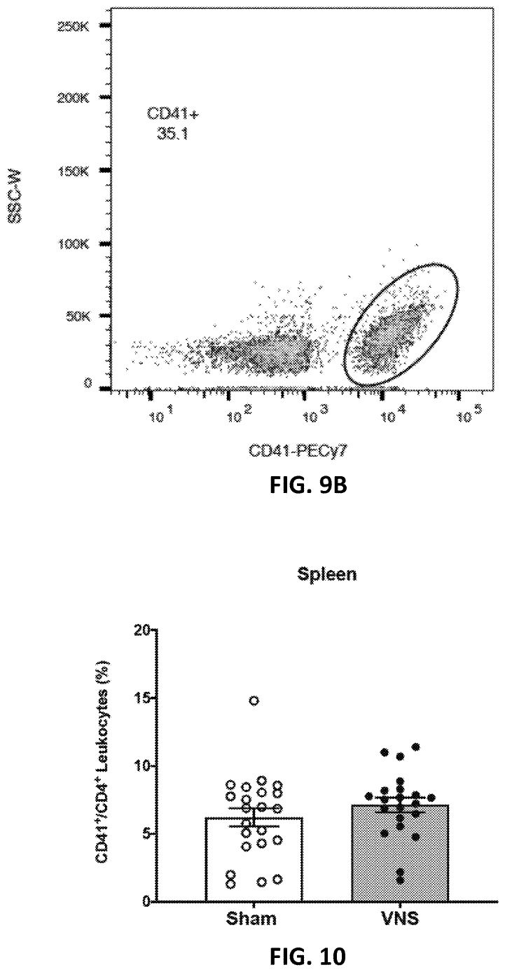

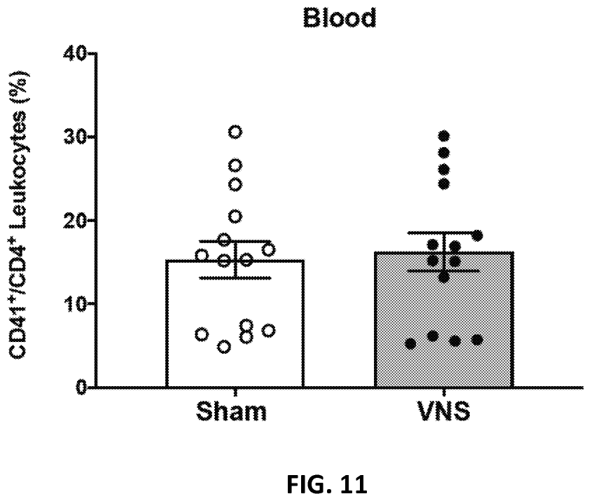

FIG. 9 illustrates a Platelet gating strategy for flow cytometry. a, Preliminary FSC/SSC gates for starting platelet cell population from murine systemic whole blood. b, Platelet cell population identification with anti-CD41a.

### Methods

### Mice

Adult male 8-12 week old BALB/c mice (20-25 g, Taconic), adult male 8-12 week old C57BL6/J mice (20-25 g, Jackson Labs), adult male 8-16 week old a7nAChR-deficient mice (20-25 g, Jackson Labs, C57BL6/J background) and wild-type littermates, adult male 8-12 week old (Foxn1nu) nude mice (20-25 g Taconic, BALB/c background), adult male 8-12 week old factor VIII knockout mice (20-25 g, Jackson Labs) are housed at 25° C. on a 12-hour light/dark cycle. Standard animal chow and water are freely available. All animal experiments are performed in accordance with the National Institutes of Health (NIH) Guidelines under protocols approved by the Institutional Animal Care and Use Committee of The Feinstein Institutes for Medical Research.

### Vagus Nerve Stimulation

Animals are anesthetized with ketamine (144 mg/kg, i.p.) and xylazine (14 mg/kg, i.p.). After seven minutes, animals are placed in a supine position, and a 1-2 cm ventral midline cervical skin incision is made between the mandible and sternum. The subcutaneous tissue and mandibular salivary glands are dissected and retracted laterally. The left vagus nerve is isolated from between the sternomastoid and sternohyoid muscles, dissected free from the neighboring carotid artery, and controlled with a 5-0 prolene suture. The nerve is then mounted on bipolar platinum electrodes (Plastics One). Constant voltage stimuli (1 V, 30 Hz, 2 ms pulse width) are applied for five minutes. Electrical stimuli are generated using an MP36R Data Acquisition System (Biopac Systems, Goleta, CA) attached to an out 3 low voltage stimulation adapter. Sham-stimulated animals receive cervical skin incision and dissection of the salivary glands, but the vagus nerve is neither isolated nor dissected free from neighboring structures.

### Tail Transection and Hemorrhage

Mice are screened before study inclusion to ensure visual similarity and consistency between the anatomy of the tail tips and the site of transection. Any animals displaying aberrant anatomy and/or injury that does not meet our predetermined criteria are excluded. Following vagus nerve stimulation or sham surgery, tails are immersed in water at 37±1° C. for five minutes. Tails are then removed from the solution, amputated 2 mm from the tip with a razor blade, and immediately placed into a 50 mL beaker containing water at 37±1° C. Tails are allowed to bleed uncontrolled until bleeding stops for a minimum of ten seconds. This duration of bleeding is recorded as bleeding time. For analyses of bleeding in factor VIII deficient mice, tails are first immersed in 0.9% saline at 37±1° C. for five minutes. Tails are then removed and amputated 2 mm from the tip with a razor blade. Tails are then placed into a 50 ml conical tube containing 0.9% saline at 37=1° C. Tails are allowed to bleed freely for a total of ten minutes. Total blood loss is determined by densitometry by measuring absorbance at 550 nm. A standard curve is created from a known volume of blood.

### Ferric Chloride Induced Carotid Artery Injury

Animals are anesthetized with isoflurane (1-2%), placed in the supine position, and a 1-2 cm ventral midline cervical skin incision is made between the mandible and sternum. The left vagus nerve is isolated from the carotid sheath and placed on a bipolar electrode and stimulated (1 V, 30 Hz, 2 ms pulse width, 5 minutes). Following stimulation or sham surgery, the right carotid artery is dissected free from surrounding tissue for a distance of 5 mm, and the artery is bathed in 5 μL of a 10% ferric chloride (FeCl3) solution for 3 minutes (F8KO mice) or a 5% FeCl3 solution for 1 minute (C57BL6/J and a7KO mice). The carotid artery is then rinsed with 0.9% normal saline. A doppler ultrasound probe (L8-18i-RS, GE) attached to a GE Logiq e ultrasound system is placed over both carotid arteries to record blood flow for a total of 25 minutes. Experimental end points include cessation of blood flow for >1 minute as determined by absence of arterial waveform on motion mode (m-mode) and doppler signal as compared to the contralateral, unaffected carotid artery. If occlusion is not observed after 25 minutes, the time is recorded as 25 minutes for statistical comparisons.

### Splenectomy

Animals are anesthetized with ketamine (144 mg/kg, i.p.) and xylazine (14 mg/kg, i.p.). Animals are placed in a supine position, and a 2 cm laparotomy incision is made with a scalpel. The spleen is exteriorized and the splenic artery and vein ligated with a 5-0 prolene suture. The peritoneum is closed with 5-0 Vicryl simple interrupted sutures and the skin with a 7 mm skin stapler. Sham splenectomized animals undergo midline laparotomy only. Splenectomy or sham splenectomy is performed six weeks before tail transection.

### Heart Rate Recording

Animals are anesthetized with isoflurane (3% induction, maintenance 1.5%). Animals are placed in the supine position, a 1-2 cm ventral midline cervical skin incision is made and the left vagus nerve is isolated and controlled as described above. The nerve is mounted on bipolar platinum electrodes (Plastics One) and constant voltage stimulation (1 V, 30 Hz, 2 ms pulse width) is applied for five minutes. Heart rate data is acquired at 30 Hz using the OmniPlex Neural Data Acquisition System (Plexon, Inc.). Data is analyzed offline using Spike2 software (Cambridge Electronic Design Ltd.). Heart rate is calculated as beats per minute (BPM). Pre-stimulation is defined as a 20 second epoch beginning 5 minutes after transition to maintenance anesthesia. Post-stimulation is defined as a 20 second epoch beginning 1 minute after vagus nerve stimulation. Pre- and post-stimulation heart rates are compared using Mann-Whitney U-test.

### Platelet Priming

Systemic blood from the inferior vena cava is collected 17 minutes after vagus nerve stimulation (5 minutes) into Tris buffered saline (20 mM Tris-HCl, 137 mM NaCl, pH 7.3) containing 20 U/mL heparin. Heparinized whole blood is then diluted with modified Tyrode's Buffer (134 mM NaCl, 0.34 mM Na2HPO4, 2.9 mM KCl, 12 mM NaHCO₃20 mM Hepes, pH 7.0 with 5 mM glucose, 0.35% BSA). On flow cytometry (BD FACSymphony A3, BD LSRFortessa), platelets are initially identified via forward and side scatter plots (FSC-A/SSC-A) followed by anti-CD41a marker (BioLegend) (FIGS. 9A-9B). Platelets are then stimulated ex vivo with thrombin (1 U/ml, Sigma-Aldrich), collagen (0.5 or 5 μg/ml, Sigma-Aldrich), or ADP (20 μg/ml Thermo Scientific), and stained with antibodies against CD62P (P-Selectin) (BioLegend), active GPIIb/IIIa (Emfret Analytics), and phosphatidylserine (Millipore Sigma). In separate experiments, non-stimulated platelets are stained with anti-nicotinic acetylcholine receptor a7 (CHRNA7) (extracellular)-FITC antibody (Alomone Labs). Flow cytometry data is then collected (BD FACSDiva Software) and analyzed (FlowJo 10.8.1).

### Thrombin Generation

Animals receive vagus nerve stimulation followed by tail warming in a 37° C. water bath for five minutes. The tail is transected where its diameter was 3 mm, and allowed to bleed freely into citrated tubes to collect local shed blood. Systemic blood from the inferior vena cava is then collected directly into citrated tubes. Blood is centrifuged for 15 minutes at 1500 g and plasma stored at −80° C. until assay. Thrombin generation is determined by measurement of thrombin-antithrombin (TAT) complex via commercially available ELISA (Abcam, Cambridge, MA).

### Factor VIII Activity

Animals receive vagus nerve stimulation (5 minutes) or sham stimulation followed by tail warming in a 37° C. water bath for five minutes. Systemic blood is collected via cardiac puncture after 7 minutes. Blood is anticoagulated with sodium citrate and centrifuged at 1500 g for 15 min. Platelet poor plasma is stored at −80° C. until assay. Factor VIII activity is determined via standard clinical assay.

### Lung Histology

Following tail transection and bleeding time measurement, lung tissue is collected. Briefly, lungs are inflated with 10% formalin by atmospheric pressure, excised, embedded in paraffin, sectioned at 6-7 μm and stained with hematoxylin and eosin. A board-certified thoracic pathologist blinded to the treatment assesses histologic sections for the presence of microthrombi and inflammation. Pictomicrographs are collected with a Zeiss Apotome connected to a computer with Zeiss AxioVision installed (Carl Zeiss Microscopy GmbH, Jena, Germany).

### Platelet Adoptive Transfer

Male aged 8-12 week old C57BL/6 mice (Taconic Biosciences, Hudson, NY) are anesthetized with ketamine/xylazine and receive vagus nerve stimulation (5 minutes) or sham stimulation as described above. After 17 minutes, systemic blood is collected from the inferior vena cava into Tris buffer saline containing 20 U/mL Heparin. Heparinized blood is centrifuged for five minutes at 500 g. Platelet rich plasma (PRP) is transferred to a clean tube and centrifuged again for eight minutes at 300 g. PRP of all mice from each stimulation group is combined and centrifuged for five minutes at 1300 g. The platelet pellet is suspended in 0.9% normal saline with 5% BSA. Final platelet concentration is determined by flow cytometry. Male aged 8-12 week old C57BL/6 mice are anesthetized with ketamine/xylazine, receive donor platelets (6×108 platelets/200 μL, r.o.), and after nine minutes undergo tail transection.

### ChAT-eGFP+ T-cell Adoptive Transfer

Transgenic ChATBAC-eGFP mice (The Jackson Laboratory, Bar Harbor, ME) are euthanized via CO2 asphyxiation followed by splenic harvest. Spleens are passed through a 40 μm cell strainer, followed by red blood cell lysis with ACK buffer (Lonza, Allendale, NJ), and then passage through a CD4+ negative selection column (Miltenyi Biotech, Bergisch Gladbach, Germany). Eluted cells are treated with Fc block, anti-CD19 (BD Pharmingen, San Jose, CA) and anti-CD62L (eBioscience, San Diego, CA). Stained cells are then negative selected for these markers by FACS where this sub-population is divided into ChAT-eGFP+ and ChAT-eGFP−. Each population is suspended in 0.9% normal saline with 5% BSA and 150,000 cells are injected into the peritoneum of male aged 8-10 week old BALB/Nu mice (Taconic Biosciences, Hudson, NY). After 5 days, recipient nude mice receive vagus nerve stimulation (5 minutes) or sham stimulation before tail transection.

### Platelet Ca2+ Flux

Male aged 8-12 week old C57BL/6 mice (Taconic Biosciences, Hudson, NY) are anesthetized with ketamine/xylazine and receive vagus nerve stimulation (1 V, 30 Hz, 2 ms, 5 minutes) or sham stimulation as described above. Harvested circulating platelets are washed and loaded with Oregon Green BAPTA-1, AM and FuraRed, AM. Platelets are resuspended in Tyrode's buffer containing 2 mM Ca2+ and baseline calcium concentrations are measured by flow cytometry.

### Cell Counts

Animals are anesthetized with ketamine (144 mg/kg, i.p.) and xylazine (14 mg/kg, i.p.) as described above. Circulating blood from the inferior vena cava is collected seven minutes after vagus nerve stimulation or sham stimulation, placed into standard EDTA tubes, and complete blood count is determined using a Cell-Dyn 3700 Blood Analyzer.

### Statistical Analysis

All data are presented as mean±s.e.m, and p<0.05 is considered significant. n represents the number of animals in each experiment, as detailed in the figure legends. Animals were randomly allocated into experimental groups. All measurements were taken from distinct samples, where appropriate. Statistical significance was determined by one-way ANOVA (Tukey) for three groups or unpaired, two-tailed Student's t-test for two groups. Statistical analyses were performed, and data presented using GraphPad Prism 8 (GraphPad Software), BD FACSDiva software (BD Biosciences), and FlowJo 10 (LLC).

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.5% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of improving platelet function of a blood donor for a hemophiliac subject, which comprises stimulating the vagus nerve of the blood donor to increase calcium uptake of the donor's platelets, wherein the blood donor has not been administered a clotting factor within the last 48 hours.

2. The method of claim 1, wherein the blood donor has not been administered the clotting factor within the last 12 hours.

3. The method of claim 1, wherein stimulating the vagus nerve activates the donor's platelet α7nAChR.

4. The method of claim 1, further comprising transferring calcium-primed platelets from the blood donor to the hemophiliac subject.

5. The method of claim 1, wherein stimulating the vagus nerve of the blood donor does not cause systemic platelet activation in the blood donor.

6. The method of claim 1, wherein stimulating the vagus nerve of the blood donor does not cause increased systemic thrombin levels in the blood donor.

7. The method of claim 1, wherein the blood donor is not hemophilic.

8. The method of claim 1, wherein the clotting factor is a factor VIII concentrate.

9. The method of claim 1, wherein calcium-primed platelets of the blood donor increase platelet secretion of hemostatic factors in the hemophiliac subject after transfer of the calcium-primed platelets from the blood donor to the hemophiliac subject.

10. The method of claim 1, wherein stimulating the vagus nerve of the blood donor increases calcium uptake of the blood donor's platelets by activating acetylcholine-producing T lymphocytes in the blood donor's spleen, leading to acetylcholine-dependent activation of platelet α7nAChR and increased blood donor platelet calcium uptake.

11. A method of increasing platelet activation of a blood donor for a hemophiliac subject, the method comprising stimulating the vagus nerve of the blood donor to increase platelet intracellular calcium and/or activate splenic acetylcholine-synthesizing T lymphocytes in the blood donor, wherein the blood donor has not been administered a clotting factor within the last 48 hours.

12. The method of claim 11, wherein stimulating the vagus nerve of the blood donor does not cause systemic platelet activation in the blood donor.

13. The method of claim 11, wherein stimulating the vagus nerve of the blood donor does not cause increased systemic thrombin levels in the blood donor.

14. The method of claim 11, wherein the blood donor has not been administered the clotting factor within the last 12 hours.

15. The method of claim 11, wherein stimulating the vagus nerve activates the donor's platelet α7nAChR.

16. The method of claim 11, wherein the clotting factor is a factor VIII concentrate.

17. A method of increasing platelet activation of a blood donor for a hemophiliac subject, the method comprising:
stimulating the vagus nerve of the blood donor to activate acetylcholine-producing T lymphocytes in the donor's spleen, leading to acetylcholine-dependent activation of platelet α7nAChR and increased blood donor platelet calcium uptake, wherein the blood donor has not been administered a clotting factor within the last 48 hours.

18. The method of claim 17, wherein stimulating the vagus nerve of the blood donor does not cause systemic platelet activation in the blood donor.

19. The method of claim 17, wherein stimulating the vagus nerve of the blood donor does not cause increased systemic thrombin levels in the blood donor.

20. The method of claim 17, wherein the blood donor is not hemophilic.
